(19) United States
(12) Patent Application Publication
(10) Pub. No.: US 2002/0159978 A1
(43) Pub. Date: Oct. 31, 2002

(54) Muscle-directed gene transfer by use of recombinant AAV-1 and AAV-6 virions

(76) Inventor: James Allen, Alameda, CA (US)

Correspondence Address: KNOBBE MARTENS OLSON & BEAR LLP, 2040 MAIN STREET, FOURTEENTH FLOOR, IRVINE, CA, 91614, US

(21) Appl. No.: 10/056,788

(22) Filed: Jan. 23, 2002

Related U.S. Application Data
(60) Provisional application No. 60/266,778, filed on Feb. 6, 2001.

(51) Int. Cl. A61K 48/00; C12N 15/861

(52) U.S. Cl. 424/93.2; 435/456; 514/44

(57) ABSTRACT

Methods for using novel recombinant adeno-associated virus (rAAV) virion serotypes are disclosed. The methods enable an increase in transduction efficiency of rAAV virions in mammalian muscle cells or tissue. Specifically, the methods described herein employ rAAV-1 and rAAV-6 serotype virions to deliver heterologous nucleic acid molecules of interest to muscle cells or tissue of a mammal. The disclosed methods describe direct injection into muscle tissue, intravascular administration of rAAV virions, and limb perfusion to deliver heterologous nucleic acid molecules of interest to at least one muscle cell of a mammal. The disclosed methods also describe the treatment of hemophilia, using the rAAV virions of the invention, by administering the rAAV virions to a mammalian subject with hemophilia so that blood coagulation proteins, such as Factor VIII or Factor IX, are expressed at levels greater than those achieved using the rAAV-2 serotype.

### CROSS REFERENCE TO RELATED APPLICATIONS

This application claims priority under 37 C.F.R. § 119(e) to Provisional Application Ser. No. 60/266,778 filed on Feb. 6, 2001, herein incorporated by reference in its entirety.

### FIELD OF THE INVENTION

The present invention relates to methods of delivering recombinant adeno-associated virus (rAAV) virions to a mammalian subject. More specifically, the invention relates to methods in which rAAV-1 and/or rAAV-6 virions are introduced into the muscle cells or tissue of a mammalian subject, including a human, to deliver therapeutic nucleic acids.

### BACKGROUND OF THE INVENTION

Scientists are continually discovering genes that are associated with human diseases such as diabetes, hemophilia, and cancer. Research efforts have also uncovered genes, such as erythropoietin (which increases red blood cell production), that are not associated with genetic disorders but instead code for proteins that can be used to treat numerous diseases. Despite significant progress in the effort to identify and isolate genes, however, a major obstacle facing the biopharmaceutical industry is how to safely and persistently deliver therapeutically effective quantities of gene products to patients.

Generally, the protein products of these genes are synthesized in cultured bacterial, yeast, insect, mammalian, or other cells and delivered to patients by direct injection. Injection of recombinant proteins has been successful but suffers from several drawbacks. For example, patients often require weekly, and sometimes daily, injections in order to maintain the necessary levels of the protein in the bloodstream. Even then, the concentration of protein is not maintained at physiological levels—the level of the protein is usually abnormally high immediately following the injection, and far below optimal levels prior to the injection. Additionally, injected delivery of recombinant protein often cannot deliver the protein to the target cells, tissues, or organs in the body. And, if the protein successfully reaches its target, it may be diluted to a non-therapeutic level. Furthermore, the method is inconvenient and often restricts the patient's lifestyle.

These shortcomings have fueled the desire to develop gene therapy methods for delivering sustained levels of specific proteins into patients. These methods are designed to allow clinicians to introduce deoxyribonucleic acid (DNA) coding for a nucleic acid, such as a therapeuic gene, directly into a patient (in vivo gene therapy) or into cells isolated from a patient or a donor (ex vivo gene therapy). The introduced nucleic acid then directs the patient's own cells or grafted cells to produce the desired protein product. Gene delivery, therefore, obviates the need for frequent injections. Gene therapy may also allow clinicians to select specific organs or cellular targets (e.g., muscle, blood cells, brain cells, etc.) for therapy.

DNA may be introduced into a patient's cells in several ways. There are transfection methods, including chemical methods such as calcium phosphate precipitation and liposome-mediated transfection, and physical methods such as electroporation. In general, transfection methods are not suitable for in vivo gene delivery. There are also methods that use recombinant viruses. Current viral-mediated gene delivery vectors include those based on retrovirus, adenovirus, herpes virus, pox virus, and adeno-associated virus (AAV). Like the retroviruses, and unlike adenovirus, AAV has the ability to integrate its genome into a host cell chromosome.

### Adeno-associated Virus-mediated Gene Therapy

AAV is a parvovirus belonging to the genus Dependovirus, and has several attractive features not found in other viruses. For example, AAV can infect a wide range of host cells, including non-dividing cells. AAV can also infect cells from different species. Importantly, AAV has not been associated with any human or animal disease, and does not appear to alter the physiological properties of the host cell upon integration. Furthermore, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

In nature, AAV is a helper virus-dependent virus, i.e., it requires co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To construct infectious recombinant AAV (rAAV) containing a nucleic acid, a suitable host cell line is transfected with an AAV vector containing a nucleic acid. AAV helper functions and accessory functions are then expressed in the host cell. Once these factors come together, the HNA is replicated and packaged as though it were a wild-type (wt) AAV genome, forming a recombinant virion. When a patient's cells are infected with the resulting rAAV, the HNA enters and is expressed in the patient's cells. Because the patient's cells lack the rep and cap genes, as well as the adenovirus accessory function genes, the rAAV are replication defective; that is, they cannot further replicate and package their genomes. Similarly, without a source of rep and cap genes, wtAAV cannot be formed in the patient's cells.

There are six known AAV serotypes, AAV-1 through AAV-6. Of those six serotypes, AAV-2 is the best characterized, having been used to successfully deliver transgenes to several cell lines, tissue types, and organs in a variety of in vitro and in vivo assays. The six serotypes of AAV can be distinguished from one another by the use of monoclonal antibodies or by employing nucleotide sequence analysis; AAV-1, AAV-2, AAV-3, and AAV-6 are 82% identical at the nucleotide level, while AAV-4 is 75 to 78% identical to the other serotypes (Russell et al. (1998) J Virol 72:309-319). Significant nucleotide sequence variation is noted for regions of the AAV genome that code for capsid proteins; such variable regions may be responsible for differences in serological reactivity to the capsid proteins of the various AAV serotypes.

It is known that readministration of a single AAV serotype can lead to a significant reduction in transduction efficiency. Moskalenko et al. (J Virol (2000) 74:176101766), for example, showed that mice with pre-existing anti-AAV-2 antibodies, when administered Factor IX in a recombinant AAV-2 virion, failed to express the Factor IX transgene, suggesting that the anti-AAV-2 antibodies blocked transduction of the rAAV-2 virion. Halbert et al. (J Virol (1998) 72:9795-9805) reported similar results. Others have demonstrated successful readministration of rAAV-2 virions into experimental animals, but only after immune suppression is achieved (e.g., Halbert et al., supra).

Thus, using rAAV-2 for human gene therapy is potentially problematic because anti-AAV-2 antibodies are prevalent in human populations; in fact, one study estimated that at least 80% of the general population has been infected with AAV-2 (Berns and Linden (1995) Bioessays 17:237-245). The identification of AAV serotypes that are not serologically cross-reactive with AAV-2 would be a significant advancement in the art. Such AAV serotypes are described herein.

### SUMMARY OF THE INVENTION

The present invention provides AAV serotypes that have the ability to efficiently transduce cell and tissue types that AAV-2 transduces poorly and/or will not be inhibited by anti-AAV-2 antibodies. In accordance with the present invention, methods and AAV vectors for use therein are provided for the efficient delivery of a heterologous nucleic acid molecule(s) (HNA) to cells or tissue of a mammal, using recombinant AAV virions. Preferably, the cells or tissue are muscle cells or muscle tissue.

More specifically, the present invention provides for the use of AAV-1 and AAV-6 serotypes (i.e., AAV virions containing AAV-1 and/or AAV-6 capsid proteins) to deliver an HNA encoding anti-sense RNA, ribozymes, or genes that express proteins, wherein expression of said anti-sense RNA, ribozymes, or genes provides for a therapeutic effect in a mammalian subject. In one embodiment, the rAAV virions containing an HNA are injected directly into a muscle. In another embodiment, the rAAV virions containing an HNA are administered into the vasculature. via injection into veins, arteries, or other vascular conduits, or by using techniques such as isolated limb perfusion.

In a preferred embodiment of the invention, AAV-1-derived and AAV-6-derived virions are provided that contain a gene encoding a blood coagulation protein which, when expressed at a sufficient concentration, provides for a therapeutic effect, such effect being an improvement in the blood-clotting efficiency of a mammal suffering from a blood clotting disorder. The blood clotting disorder can be any disorder adversely affecting the organism's ability to coagulate the blood. Preferably, the blood clotting disorder is hemophilia.

In one embodiment, the gene encoding a blood coagulation protein is a Factor VIII gene. Preferably, the Factor VIII gene is the human Factor VIII gene or a derivation thereof. In another embodiment, the gene encoding a blood coagulation protein is a Factor IX gene. Preferably, the Factor IX gene is the human Factor IX (hF.IX) gene.

These and other embodiments of the instant invention will readily occur to those of ordinary skill in the art in view of the disclosure herein.

### BRIEF DESCRIPTION OF THE DRAWINGS

FIG. 1 represents circulating plasma hF.IX in nanograms per milliliter (ng/mL) as measured in RAG-1 mice following intramuscular (IM) injection of 2×10¹¹ viral vector genomes/kg (n=4).

### DETAILED DESCRIPTION OF THE INVENTION

The present invention embraces the use of a recombinant adeno-associated virus (rAAV) virion to deliver a “heterologous nucleic acid” (an “HNA”)to a mammalian subject. A “recombinant AAV virion” or “rAAV virion” is an infectious virus composed of an AAV protein shell (i.e., a capsid) encapsulating a “recombinant AAV (rAAV) vector,” the rAAV vector comprising the HNA and one or more AAV inverted terminal repeats (ITRs). AAV vectors can be constructed using recombinant techniques that are known in the art and include one or more HNAs flanked by functional ITRs. The ITRs of the rAAV vector need not be the wild-type nucleotide sequences, and may be altered, e.g., by the insertion, deletion, or substitution of nucleotides, so long as the sequences provide for proper function, i.e., rescue, replication, and packaging of the AAV genome.

Recombinant AAV virions may be produced using a variety of techniques known in the art, including the triple transfection method (described in detail in U.S. Pat. No. 6,001,650, the entirety of which is incorporated by reference). This system involves the use of three vectors for rAAV virion production, including an AAV helper function vector, an accessory function vector, and a rAAV vector that contains the HNA. One of skill in the art will appreciate, however, that the nucleic acid sequences encoded by these vectors can be provided on two or more vectors in various combinations. As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). An example of such a vector, pHLP19 is described in U.S. Pat. No. 6,001,650. Another AAV helper function vector is the pRep6cap6 vector, described in U.S. Pat. No. 6,156,303, the entirety of which is herein incorporated by reference.

The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. In a preferred embodiment, the accessory function plasmid pladeno5 is used (details regarding pLadeno5 are described in U.S. Pat. No. 6,004,797, the entirety of which is hereby incorporated by reference). This plasmid provides a complete set of adenovirus accessory functions for AAV vector production, but lacks the components necessary to form replication-competent adenovirus.

The instant invention broadly contemplates the use of two specific AAV serotypes: AAV-1 and the AAV-6. The term “serotype” is used herein to describe the genotype of a virus or unicellular organism that has been defined by means of antisera binding to antigenic determinants located on the surface of the virus or unicellular organism. In the case of AAV, the antigenic determinants located on the AAV virion's surface are the capsid proteins, so it is the capsid protein that distinguishes the AAV serotype. Capsid proteins are the product of AAV cap gene expression, the specific sequence of the cap gene being unique to the particular AAV serotype.

Recombinant AAV-6 virions are produced, in one embodiment of the invention, with an AAV helper function vector containing the rep and cap genes from the AAV-6 genome (this vector is known as pRepCap6—see U.S. Pat. No. 6,156,303, supra, for a thorough description of the pRepCap6 vector). The AAV-6 genome has been published and is available under GenBank Accession No. 9629894. One of skill in the art will also appreciate that other rep genes, e.g. rep2, can be used in combination with the cap6 gene to produce AAV “hybrid” helper function vectors, which are then capable of supporting the production of rAAV-6 virions. The term “hybrid” as used herein is an AAV helper function vector with a rep gene from one serotype (other than AAV-6) in combination with the cap gene from the AAV-6 genome (e.g., pRep2Cap6, pRep3Cap6, etc.). The rep and cap genes can be wild-type in their sequences, or be altered by partial deletion, mutation, rearrangement, addition, gene or gene segment shuffling, etc., the primary consideration as contemplated herein being the retention of rep and cap wild-type function. See U.S. Pat. No. 6,156,303, supra, (and Example 1 below) for methods describing the generation of rAAV-6 virions.

Similar methods can also be employed to construct AAV helper function vectors containing rep and cap1 genes. Incorporating the rep gene from the AAV-1 genome with the cap gene from the AAV-1 genome yields the AAV helper function vector pRepCap1. Incorporating a rep gene from an AAV serotype other than AAV-1 yields a hybrid AAV helper function vector still capable of supporting AAV-1 virion production since the AAV helper function vector contains the cap1 gene. Either pRepCap1 or a hybrid AAV helper function vector such as pRep2Cap1 can support rAAV-1 virion production. The AAV-1 genome has been published under the Pat. Cooperation Treaty (international publication WO 0028061) and is available under GenBank Accession No. 9632547.

We have shown that the cap 1 and cap6 proteins recognize the cap binding site(s) on the AAV-2 ITRs. This is thought to be because the cap 1 and cap6 proteins recognize the AAV-2 ITR secondary structure, and not specific AAV-2 ITR DNA sequences. It is believed that the ITRs from the various AAV serotypes assume similar secondary structure so one of skill in the art would appreciate that AAV-1, AAV-3, AAV-4, AAV5, or AAV-6 ITRs could be used with pRep6Cap6 or a hybrid AAV-6 helper function vector to generate AAV-6 serotype virions (or pRep1Cap1 or a hybrid AAV-1 helper function vector to generate AAV-1 serotype virions). For example, using the methods of the instant invention, pRep2Cap6 could be used in conjunction with AAV-3 ITRs to produce AAV-6 virions.

The HNA, that is, the “heterologous nucleic acid,” comprises nucleic acid sequences joined together that are otherwise not found together in nature, this concept defining the term “heterologous.” To illustrate the point, an example of an HNA is a gene flanked by nucleotide sequences not found in association with that gene in nature. Another example of an HNA is a gene that itself is not found in nature (e.g., synthetic sequences having codons different from the native gene). Allelic variation or naturally occurring mutational events do not give rise to HNAs, as used herein. An HNA can comprise an anti-sense RNA molecule, a ribozyme, or a gene encoding a polypeptide.

The HNA is operably linked to a heterologous promoter (constitutive, cell-specific, or inducible) such that the HNA is capable of being expressed in the patient's target cells under appropriate or desirable conditions. Numerous examples of constitutive, cell-specific, and inducible promoters are known in the art, and one of skill could readily select a promoter for a specific intended use, e.g., the selection of the muscle-specific skeletal α-actin promoter or the muscle-specific creatine kinase promoter/enhancer for muscle cell-specific expression, the selection of the constitutive CMV promoter for strong levels of continuous or near-continuous expression, or the selection of the inducible ecdysone promoter for induced expression. Induced expression allows the skilled artisan to control the amount of protein that is synthesized. In this manner, it is possible to vary the concentration of therapeutic product. Other examples of well known inducible promoters are: steroid promoters (e.g., estrogen and androgen promoters) and metallothionein promoters.

The invention includes rAAV-1 or rAAV-6 virions comprising HNAs coding for one or more anti-sense RNA molecules, the rAAV virions preferably administered to one or more muscle cells or tissue of a mammal. Antisense RNA molecules suitable for use with the present invention in cancer anti-sense therapy or treatment of viral diseases have been described in the art. See, e.g., Han et al., (1991) Proc. Natl Acad. Sci. USA 88:4313-4317; Uhlmann et al., (1990) Chem. Rev. 90:543-584; Helene et al., (1990) Biochim. Biophys. Acta. 1049:99-125; Agarawal et al., (1988) Proc. Natl. Acad. Sci. USA 85:7079-7083; and Heikkila et al., (1987) Nature 328:445-449. The invention also encompasses the delivery of ribozymes using the methods disclosed herein. For a discussion of suitable ribozymes, see, e.g., Cech et al., (1992) J Biol Chem. 267:17479-17482 and U.S. Pat. No. 5,225,347.

The invention preferably encompasses rAAV-1 or rAAV-6 virions comprising HNAs coding for one or more polypeptides, the rAAV virions preferably administered to one or more muscle cells or tissue of a mammal. Thus, the invention embraces the delivery of HNAs encoding one or more peptides, polypeptides, or proteins, which are useful for the treatment of disease states in a mammalian subject. Such DNA and associated disease states include, but are not limited to: DNA encoding glucose-6-phosphatase, associated with glycogen storage deficiency type 1A; DNA encoding phosphoenolpyruvate-carboxykinase, associated with Pepck deficiency; DNA encoding galactose-1 phosphate uridyl transferase, associated with galactosemia; DNA encoding phenylalanine hydroxylase, associated with phenylketonuria; DNA encoding branched chain alpha-ketoacid dehydrogenase, associated with Maple syrup urine disease; DNA encoding fumarylacetoacetate hydrolase, associated with tyrosinemia type 1; DNA encoding methylmalonyl-CoA mutase, associated with methylmalonic acidemia; DNA encoding medium chain acyl CoA dehydrogenase, associated with medium chain acetyl CoA deficiency; DNA encoding ornithine transcarbamylase, associated with ornithine transcarbamylase deficiency; DNA encoding argininosuccinic acid synthetase, associated with citrullinemia; DNA encoding low density lipoprotein receptor protein, associated with familial hypercholesterolemia; DNA encoding UDP-glucouronosyltransferase, associated with Crigler-Najjar disease; DNA encoding adenosine deaminase, associated with severe combined immunodeficiency disease; DNA encoding hypoxanthine guanine phosphoribosyl transferase, associated with Gout and Lesch-Nyan syndrome; DNA encoding biotinidase, associated with biotinidase deficiency; DNA encoding beta-glucocerebrosidase, associated with Gaucher disease; DNA encoding beta-glucuronidase, associated with Sly syndrome; DNA encoding peroxisome membrane protein 70 kDa, associated with Zellweger syndrome; DNA encoding porphobilinogen deaminase, associated with acute intermittent porphyria; DNA encoding alpha-1 antitrypsin for treatment of alpha-1 antitrypsin deficiency (emphysema); DNA encoding erythropoietin for treatment of anemia due to thalassemia or to renal failure; DNA encoding vascular endothelial growth factor, DNA encoding angiopoietin-1, and DNA encoding fibroblast growth factor for the treatment of ischemic diseases; DNA encoding thrombomodulin and tissue factor pathway inhibitor for the treatment of occluded blood vessels as seen in, for example, atherosclerosis, thrombosis, or embolisms; DNA encoding aromatic amino acid decarboxylase (AADC), and DNA encoding tyrosine hydroxylase (TH) for the treatment of Parkinson's disease; DNA encoding the beta adrenergic receptor, DNA encoding anti-sense to, or DNA encoding a mutant form of, phospholamban, DNA encoding the sarco(endo)plasmic reticulum adenosine triphosphatase-2 (SERCA2), and DNA encoding the cardiac adenylyl cyclase for the treatment of congestive heart failure; DNA encoding a tumor suppessor gene such as p53 for the treatment of various cancers; DNA encoding a cytokine such as one of the various interleukins for the treatment of inflammatory and immune disorders and cancers; DNA encoding dystrophin or minidystrophin and DNA encoding utrophin or miniutrophin for the treatment of muscular dystrophies; and, DNA encoding insulin for the treatment of diabetes.

The invention also includes rAAV-1 and rAAV-6 virions comprising a gene or genes coding for blood coagulation proteins, which proteins may be delivered, using the methods of the present invention, to the cells of a mammal having hemophilia for the treatment of hemophilia. Thus, the invention includes: delivery of the Factor IX gene to a mammal for treatment of hemophilia B, delivery of the Factor VIII gene to a mammal for treatment of hemophilia A, delivery of the Factor VII gene for treatment of Factor VII deficiency, delivery of the Factor X gene for treatment of Factor X deficiency, delivery of the Factor XI gene for treatment of Factor XI deficiency, delivery of the Factor XIII gene for treatment of Factor XIII deficiency, and, delivery of the Protein C gene for treatment of Protein C deficiency. Delivery of each of the above-recited genes to the cells of a mammal is accomplished by first generating a rAAV virion comprising the gene and then administering the rAAV virion to the mammal. Thus, the invention includes rAAV virions comprising genes encoding any one of Factor IX, Factor VIII, Factor X, Factor VII, Factor XI, Factor XIII or Protein C.

Delivery of rAAV-1 or rAAV-6 virions containing one or more HNAs to a mammalian subject may be by intramuscular injection or by administration into the bloodstream of the mammalian subject. Administration into the bloodstream may be by injection into a vein, an artery, or any other vascular conduit such as a venule, an arteriole, or capillary. Additionally, a skilled artisan can administer rAAV-1 or rAAV-6 virions into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403 and herein incorporated by reference, can also be employed by the skilled artisan to administer rAAV-1 or rAAV-6 virions into the vasculature of an isolated limb to potentially enhance transduction into muscle cells or tissue.

The dose of rAAV virions required to achieve a particular “therapeutic effect,” e.g., the units of dose in vector genomes/per kilogram of body weight (vg/kg), will vary based on several factors including, but not limited to: the route of rAAV virion administration, the level of gene (or anti-sense RNA or ribozyme) expression required to achieve a therapeutic effect, the specific disease or disorder being treated, a host immune response to the rAAV virion, a host immune response to the gene (or anti-sense RNA or ribozyme) expression product, and the stability of the gene (or anti-sense RNA or ribozyme) product. One of skill in the art can readily determine a rAAV virion dose range to treat a patient having a particular disease or disorder based on the aforementioned factors, as well as other factors that are well known in the art.

Generally speaking, by “therapeutic effect” is meant a level of expression of one or more HNAs sufficient to alter a component of a disease (or disorder) toward a desired outcome or clinical endpoint, such that a patient's disease or disorder shows clinical improvement, often reflected by the amelioration of a clinical sign or symptom relating to the disease or disorder. Using hemophilia as a specific disease example, a “therapeutic effect” for hemophilia is defined herein as an increase in the blood-clotting efficiency of a mammal afflicted with hemophilia, efficiency being determined, for example, by well known endpoints or techniques such as employing assays to measure whole blood clotting time or activated prothromboplastin time. Reductions in either whole blood clotting time or activated prothromboplastin time are indications of an increase in blood-clotting efficiency. In severe cases of hemophilia, hemophiliacs having less than 1% of normal levels of Factor VIII or Factor IX have a whole blood clotting time of greater than 60 minutes as compared to approximately 10 minutes for non-hemophiliacs. Expression of 1% or greater of Factor VIII or Factor IX has been shown to reduce whole blood clotting time in animal models of hemophilia, so achieving a circulating Factor VIII or Factor IX plasma concentration of greater than 1% will likely achieve the desired therapeutic effect of an increase in blood-clotting efficiency.

Rather than focusing exclusively on treating a disease, it is often desirable to deliver an HNA to a host cell in order to elucidate its physiological or biochemical function(s). The HNA can be either an endogenous gene or heterologous. Using either an ex vivo or in vivo approach, the skilled artisan can administer rAAV-1 and/or rAAV6 virions containing one or more HNAs of unknown function to an experimental animal, express the HNA(s), and observe any subsequent functional changes. Such changes can include: protein-protein interactions, alterations in biochemical pathways, alterations in the physiological functioning of cells, tissues, organs, or organ systems, and/or the stimulation or silencing of gene expression.

Alternatively, the skilled artisan can of over-express a gene of known function and examine its effects. Such genes can be either endogenous to the experimental animal or heterologous in nature (i.e., a transgene).

By using the methods of the present invention, the skilled artisan can also abolish or significantly reduce gene expression, thereby employing another means of determining gene function. One method of accomplishing this is by way of administering antisense RNA-containing rAAV virions to an experimental animal, expressing the antisense RNA molecule so that the targeted endogenous gene is “knocked out,” and then observing any subsequent physiological or biochemical changes.

The methods of the present invention are compatible with other well-known technologies such as transgenic mice and knockout mice and can be used to complement these technologies. One skilled in the art can readily determine combinations of known technologies with the methods of the present invention to obtain useful information on gene function.

Once delivered, in many instances it is not enough to simply express the HNA; instead, it is often desirable to vary the levels of HNA expression. Varying HNA expression levels, which varies the dose of the HNA expression product, is frequently useful in acquiring and/or refining functional information on the HNA. This can be accomplished, for example by incorporating a heterologous inducible promoter into the rAAV virion containing the HNA so that the HNA will be expressed only when the promoter is induced. Some inducible promoters can also provide the capability for refining HNA expression levels; that is, varying the concentration of inducer will fine-tune the concentration of HNA expression product. This is sometimes more useful than having an “on-off” system (i.e., any amount of inducer will provide the same level of HNA expression product, an “all or none” response). Numerous examples of inducible promoters are known in the art including the ecdysone promoter, steroid promoters (e.g., estrogen and androgen promoters) and metallothionein promoters.

The methods of the present invention can be used to facilitate pharmaco- or toxico-kinetic studies. For example, because AAV is known to transduce hepatocytes with high efficiency, human metabolic enzymes (e.g., various oxidases and reductases such as the cytochrome p450 isozymes, various epoxide hydrolases, various dehydrogenases such as alcohol and aldehyde dehydrogenases, various peptidases, etc.—metabolic enzymes that are expressed and function in hepatocytes) can be delivered to the liver of mice by way of rAAV-1 and or rAAV-6 virions, expressed, and then various drugs and/or toxicants can be administered to the transduced mice in order to screen for any metabolites of interest.

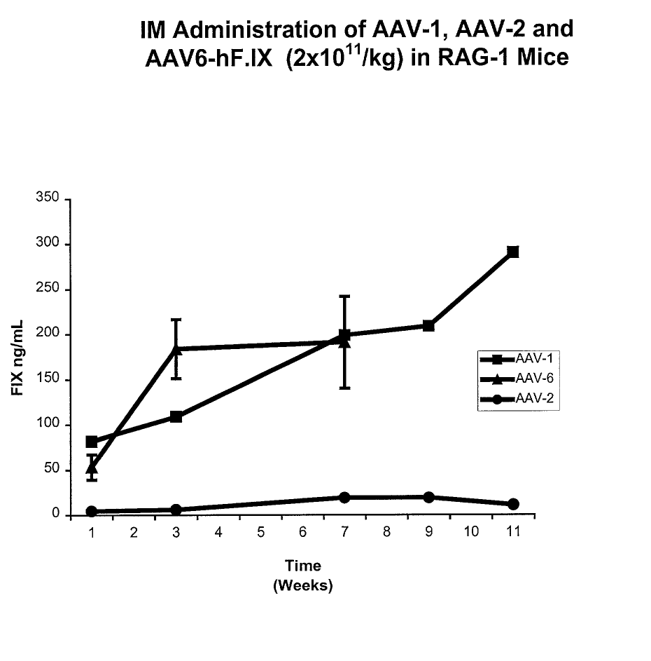

The presented methods resulted in an unexpected transduction efficiency of rAAV-6 in the skeletal muscle of mice, with transduction efficiency measured by circulating plasma levels of hF.IX (the hF.IX gene delivered to the skeletal muscle of mice by rAAV-6 virions). As shown in FIG. 1, after three weeks post-injection of rAAV-6-hF.IX, serum levels of hF.IX were approximately 32-fold greater than serum levels of hF.IX in mice injected with rAAV2-hF.IX virions. After seven weeks following injection, hF.IX delivered by rAAV-6 remained at higher concentrations than rAAV-2-delivered hF.IX. For rAAV-1-hF.IX, circulating hF.IX levels were approximately 18-fold higher than circulating F.IX levels obtained from rAAV-2-hF.IX mice. After eleven weeks post-injection, the difference between rAAV-1-hF.IX and rAAV-2-hF.IX increased to 50-fold.

The following examples are presented in order to more fully illustrate the preferred embodiments of the invention. They should in no way be construed, however, as limiting the broad scope of the invention.

### EXAMPLE 1

### Recombinant AAV Factor IX Virion Preparation

Recombinant AAV virions containing the human Factor IX (hF.IX) gene—the complete cDNA sequence for hF.IX available under GenBank Accession No. 182612—were prepared using a triple-transfection procedure described in U.S. Pat. No. 6,001,650, supra.

### Vector Construction

### AAV pRepCap6 Helper Function Vector

The pRepCap6 AAV helper function vector was constructed using standard molecular biological techniques. Using an infectious AAV-6 virion (the published wild-type sequence available under GenBank Accession No. 9629894), two Bgl II restriction sites were engineered into the AAV-6 genome, one just upstream of the p5 promoter and one downstream of the polyadenylation site, creating the pAAV6Bg1 plasmid. The Bgl II fragment containing the rep6 and cap6 genes was excised from pAAV6Bg1 and inserted into a pBLUE-SCRIPT (Stratagene, La Jolla, Calif.) backbone to create the AAV-6 helper vector pRepCap6.

### pLadeno5 Accessory Function Vector

The accessory function vector pLadeno5 was constructed as follows: DNA fragments encoding the E2a, E4, and VA RNA regions isolated from purified adenovirus serotype-2 DNA (obtained from Gibco/BRL) were ligated into a plasmid called pAmpscript. The pAmpscript plasmid was assembled as follows: oligonucleotide-directed mutagenesis was used to eliminate a 623-bp region including the polylinker and alpha complementation expression cassette from pBSII s/k+ (obtained from Stratagene), and replaced with an EcoRV site. The sequence of the mutagenic oligo used on the oligonucleotide-directed mutagenesis was 5′-CCGCTACAGGGCGCGATATCAGCTCACTCAA-3′. A polylinker (containing the following restriction sites: Bam HI; KpnI; SrfI; XbaI; ClaI; Bst1107I; SalI; PmeI; and NdeI) was synthesized and inserted into the EcoRV site created above such that the BamHI side of the linker was proximal to the f1 origin in the modified plasmid to provide the pAmpscript plasmid. The sequence of the polylinker was 5′-GGATCCGGTACCGCCCGGGCTCTAGAATCGATGTATACGTCGACGTTTAAACCATATG-3′.

DNA fragments comprising the adenovirus serotype-2 E2a and VA RNA sequences were cloned directly into pAmpscript. In particular, a 5962-bp SrfI-KpnI(partial) fragment containing the E2a region was cloned between the SrfI and KpnI sites of pAmpscript. The 5962-bp fragment comprises base pairs 21,606-27,568 of the adenovirus serotype-2 genome. The complete sequence of the adenovirus serotype-2 genome is accessible under GenBank No. 9626158.

The DNA comprising the adenovirus serotype-2 E4 sequences had to be modified before it could be inserted into the pAmpscript polylinker. Specifically, PCR mutagenesis was used to replace the E4 proximal, adenoviral terminal repeat with a SrfI site. The location of this SrfI site is equivalent to base pairs 35,836-35,844 of the adenovirus serotype-2 genome. The sequences of the oligonucleotides used in the mutagenesis were: 5′-AGAGGCCCGGGCGTTTTAGGGCGGAGTAACTTGC-3′ and 5′-ACATACCCGCAGGCGTAGAGAC-3′. A 3,192 bp E4 fragment, produced by cleaving the above-described modified E4 gene with SrfI and SpeI, was ligated between the SrfI and XbaI sites of pAmpscript which already contained the E2a and VA RNA sequences to result in the pLadeno5 plasmid. The 3,192-bp fragment is equivalent to base pairs 32,644-35,836 of the adenovirus serotype-2 genome.

### rAAV-2 hF.IX vector

The rAAV-2 hF.IX vector is an 11,442-bp plasmid containing the cytomegalovirus (CMV) immediate early promoter, exon 1 of hF.IX, a 1.4-kb fragment of hF.IX intron 1, exons 2-8 of h.FIX, 227 bp of h.FIX 3′ UTR, and the SV40 late polyadenylation sequence between the two AAV-2 inverted terminal repeats (U.S. Pat. No. 6,093,392, herein incorporated by reference). The 1.4-kb fragment of hF.IX intron 1 consists of the 5′ end of intron 1 up to nucleotide 1098 and the sequence from nucleotide 5882 extending to the junction with exon 2. The CMV immediate early promoter and the SV40 late polyadenylation signal sequences can be obtained from the published sequence of pCMV-Script®, which is available from the Stratagene catalog, Stratagene, La Jolla, Calif, and from their website, www.stratagene.com.

### Triple Transfection Procedure

Specifically, for rAAV-6 virion production, the AAV helper function rep⁶cap⁶ vector (described in U.S. Pat. No. 6,156,303, supra), the accessory function vector pLadeno5 (described in U.S. Pat. No. 6,004,797, supra), and the rAAV2-hF.IX vector (U.S. Pat. No. 6,093,392, supra) were used. Briefly, human embryonic kidney cells type 293 (293cells—available from the American Type Culture Collection, catalog number CRL-1573) were seeded in 10 cm tissue culture-treated sterile dishes at a density of 3×10⁶ cells per dish in 10 mL of cell culture medium consisting of Dulbeco's modified Eagle's medium supplemented with 10% fetal calf serum and incubated in a humidified environment at 37° C. in 5% CO₂. After overnight incubation, 293 cells were approximately eighty-percent confluent. The 293 cells were then transfected with DNA by the calcium phosphate precipitate method, a transfection method well known in the art. Briefly, 10 μg of each vector (pRepCap6, pLadeno5, and rAAV2-hF.IX) were added to a 3-mL sterile, polystyrene snap cap tube using sterile pipette tips. 1.0 mL of 300 mM CaCl₂ (JRH grade) was added to each tube and mixed by pipetting up and down. An equal volume of 2×HBS (274 mM NaCl, 10 mM KCl, 42 mM HEPES, 1.4 mM Na₂PO₄, 12 mM dextrose, pH 7.05, JRH grade) was added with a 2-mL pipette, and the solution was pipetted up and down three times. The DNA mixture was immediately added to the 293 cells, one drop at a time, evenly throughout the dish. The cells were then incubated in a humidified environment at 37° C. in 5% CO₂ for six hours. A granular precipitate was visible in the transfected cell cultures. After six hours, the DNA mixture was removed from the cells, which were then provided with fresh cell culture medium and incubated for an additional 72 hours.

After 72 hours, the cells were lysed and then treated with nuclease to reduce residual cellular and plasmid DNA. After precipitation, rAAV virions were purified by two cycles of isopycnic centrifugation; fractions containing rAAV virions were pooled, dialysed, and concentrated. The concentrated virions were formulated, sterile filtered (0.22 μM) and aseptically filled into glass vials. Vector genomes were quantified by the “Real Time Quantitative Polymerase Chain Reaction” method (Real Time Quantitative PCR. Heid C. A., Stevens J., Livak K. J., and Williams P. M. 1996. Genome Research 6:986-994. Cold Spring Harbor Laboratory Press).

Recombinant AAV1-hF.IX virions were produced in an analogous manner to rAAV6-hF.IX virions, with a pRep1Cap1 AAV helper function vector used in place of the pRepCap6 AAV helper function vector.

Recombinant AAV-2 virions were produced with the pHLP19 helper function vector (described in U.S. Pat. No. 6,001,650, supra), the pLadeno5 plasmid, and the rAAV2-hF.IX expression plasmid.

### EXAMPLE 2

Hemophilia B Treatment in RAG-1 Mice with rAAV1-HF.IX, rAAV2-HF.IX, and rAAV-6-HF.IX

RAG-1 female immunodeficient mice (homozygous for a mutation in the recombinase activating gene 1, and functionally equivalent to severe combined immunodeficiency mice because these mice do not produce mature B or T cells) 4-6 weeks old (obtained from Jackson Laboratories, Bar Harbor, ME) were injected with rAAV-6 virions (prepared as described in Example 1). Mice were anesthetized with an intraperitoneal injection of ketamine (70 mg/kg) and xylazine (10 mg/kg), and a 1 cm longitudinal incision was made in the lower extremity. Recombinant AAV6-hF.IX (2×10¹¹ viral vector genomes/kg in HEPES-Buffered-Saline, pH 7.8) virions were injected into the tibialis anterior (25 μL) and the quadriceps muscle (50 μL) of each leg using a Hamilton syringe. Incisions were closed with 4-0 Vicryl suture. Blood samples were collected at seven-day intervals from the retro-orbital plexus in microhematocrit capillary tubes and plasma assayed for hF.IX by ELISA. Human F.IX antigen in mouse plasma was assessed by ELISA as described by Walter et al. (Proc Natl Acad Sci USA (1996) 3:3056-3061). The ELISA did not cross-react with mouse F.IX. All samples were assessed in duplicate. Protein extracts obtained from injected mouse muscle were prepared by maceration of muscle in PBS containing leupeptin (0.5 mg/mL) followed by sonication. Cell debris was removed by microcentrifugation, and 1:10 dilutions of the protein extracts were assayed for hF.IX in the ELISA. The circulating plasma concentrations of hF.IX, as measured by ELISA after three weeks post-IM injection, were 185 ng/mL for rAAV-6 hF.IX gene delivery, 110 ng/mL for rAAV1-hF.IX gene delivery, and 6 ng/mL for rAAV-2 hF.IX gene delivery. After seven weeks post-injection, hF.IX plasma concentrations increased to approximately 190 ng/mL for rAAV6-hF.IX gene delivery, 200 ng/mL for rAAV1-hF.IX gene delivery, and 20 ng/mL for rAAV2-hF.IX gene delivery (see FIG. 1). After eleven weeks post-injection, hF.IX plasma concentrations increased to approximately 300 ng/mL for rAAV1-hF.IX gene delivery, but decreased to approximately 10 ng/mL for rAAV2-hF.IX gene delivery.

### EXAMPLE 3

Hemophilia B Treatment in Dogs with AAV1-cF.IX

A colony of dogs having severe hemophilia B comprising males that are hemizygous and females that are homozygous for a point mutation in the catalytic domain of the canine factor IX (cF.IX) gene, was used to test the efficacy of cF.IX delivered by rAAV-1 virions (rAAV1-cF.IX). The severe hemophilic dogs lack plasma cF.IX, which results in an increase in whole blood clotting time (WBCT) to >60 minutes (normal dogs have a WBCT between 6-8 minutes), and an increase in activated partial thromboplastin time (aPTT) to 50-80 seconds (normal dogs have an aPTT between 13-18 seconds). These dogs experience recurrent spontaneous hemorrhages. Typically, significant bleeding episodes are successfully managed by the single intravenous infusion of 10 mL/kg of normal canine plasma; occasionally, repeat infusions are required to control bleeding.

Under general anesthesia, hemophilia B dogs were injected intramuscularly with rAAV1-cF.IX virions at a dose of 1×10¹² vg/kg. The animals were not given normal canine plasma during the procedure.

Whole blood clotting time was assessed for cF.IX in plasma. Activated partial thromboplastin time was measured. A coagulation inhibitor screen was also performed. Plasma obtained from a treated hemophilic dog and from a normal dog was mixed in equal volumes and was incubated for 2 hours at 37° C. The inhibitor screen was scored as positive if the aPTT clotting time was 3 seconds longer than that of the controls (normal dog plasma incubated with imidazole buffer and pre-treatment hemophilic dog plasma incubated with normal dog plasma). Neutralizing antibody titer against AAV vector was assessed.

In the hemophilia B dogs injected with AAV1-cF.IX, WBCT was shortened from>60 min to 13 min (normal: 12-15 min).

### EXAMPLE 4

Hemophilia B Treatment in Humans with AAV6-HF.IX

On Day 0 of the protocol patients are infused with HF.IX concentrate to bring factor levels up to ˜100%, and, under ultrasound guidance, rAAV6-h.FIX virions are injected directly into 10-12 sites in the vastus lateralis of either or both anterior thighs. Injectate volume at each site is 250-500 μL, and sites are at least 2 cm apart. Local anesthesia to the skin is provided by ethyl chloride or eutectic mixture of local anesthetics. To facilitate subsequent muscle biopsy, the skin overlying several injection sites is tattooed and the injection coordinates recorded by ultrasound. Patients are observed in the hospital for 24 h after injection; routine isolation precautions will be observed during this period to minimize any risk of horizontal transmission of virions. Patients are discharged and seen daily in outpatient clinic daily for three days after discharge, then weekly at the home hemophilia center for the next eight weeks, then twice monthly up to five months, them monthly for the remainder of the year, then annually in follow-up. Circulating plasma levels of hF.IX are quantified using ELISA as described in Example 2.

What is claimed is:

1. A method of treating hemophilia in a mammal, comprising:
providing at least one recombinant adeno-associated virus (rAAV) virion, said rAAV virion comprising an AAV-6 capsid, and a heterologous nucleic acid encoding Factor IX operably linked to expression control elements; and
administering said rAAV virion to at least one muscle cell of a mammal wherein said Factor IX is expressed at levels having a therapeutic effect on said mammal, wherein said therapeutic effect is an increase in blood-clotting efficiency in said mammal.

2. The method of claim 1, wherein said Factor IX is human Factor IX.

3. A method of delivering a heterologous nucleic acid to at least one muscle cell in a mammalian subject, comprising:
(a) providing at least one recombinant adeno-associated virus (rAAV) virion, said rAAV virion comprising an AAV-6 capsid and a heterologous nucleic acid operably linked to expression control elements; and
(b) administering said rAAV virions to said muscle cell, whereby expression of said heterologous nucleic acid provides for a therapeutic effect.

4. The method of claim 3, wherein said heterologous nucleic acid is a gene encoding a protein.

5. The method of claim 3, wherein said heterologous nucleic acid is an antisense RNA.

6. The method of claim 3, wherein said heterologous nucleic acid is a ribozyme.

7. The method of claim 4, wherein said protein is a secreted protein.

8. The method of claim 7, wherein said secreted protein is a blood coagulation factor.

9. The method of claim 8, wherein said blood coagulation factor is human factor IX.

10. The method of claim 3, wherein said administering of said rAAV virions is by way of direct injection to said muscle cell of said mammalian subject.

11. The method of claim 10, wherein said muscle cell is a skeletal muscle cell.

12. The method of claim 3, wherein said administering of said rAAV virions is by way of administration to a vascular conduit of said mammalian subject.

13. The method of claim 13, wherein said vascular conduit is a vein.

14. The method of claim 13, wherein said vascular conduit is an artery.

15. The method of claim 3, wherein said therapeutic effect is an increase in blood-clotting efficiency in said mammalian subject.
